(19) United States
(12) Patent Application Publication
(10) Pub. No.: US 2010/0136121 A1
(43) Pub. Date: Jun. 3, 2010

(54) MEDICAMENTS

(75) Inventor: Mark Sanders, St. Albans (GB)

Correspondence Address: NIXON PEABODY LLP - PATENT GROUP, 1100 CLINTON SQUARE, ROCHESTER, NY, 14604, US

(73) Assignee: INNOVATA BIOMED LIMITED, St. Albans (GB)

(21) Appl. No.: 12/683,959

(22) Filed: Jan. 7, 2010

Related U.S. Application Data
(63) Continuation of application No. 11/511,730, filed on Aug. 29, 2006, now abandoned.
Application No. 11/511,730 is a continuation of application No. 10/467,153, filed on Dec. 8, 2003, now abandoned.

(30) Foreign Application Priority Data
Feb. 6, 2001 (GB) 0102902.4
Apr. 12, 2001 (GB) 0109215.4

(51) Int. Cl. A61K 9/14; A61K 31/56; A61K 9/12; A61M 15/00

(52) U.S. Cl. 424/489; 514/171; 424/45; 128/203.15

(57) ABSTRACT

There is described a bimodal pharmaceutical composition comprising effective amounts of a first active ingredient which substantially comprises a coarse fraction and a second active ingredient which substantially comprise a fine fraction characterized in that the coarse fraction possesses a greater mass median aerodynamic diameter than the fine fraction. There is also described a method of delivering a therapeutically effective amount of a substantially fine active ingredient to the lung of a patient by co-administration with a substantially coarse active ingredient.

This application is a continuation of U.S. patent application Ser. No. 11/511,730, filed Aug. 29, 2006, which is a continuation of U.S. patent application Ser. No. 10/467,153, which is the U.S. national stage patent application based on PCT International Application No. PCT/GB02/00480, filed Feb. 5, 2002, which claims the benefit of Great Britain Patent Application Serial Nos. 0102902.4 and 0109215.4, filed Feb. 6, 2001 and Apr. 12, 2001, respectively, all of which are hereby incorporated by reference in their entirety.

This invention relates to a novel medicament, novel formulations comprising the medicament and novel methods of treatment.

UK Patent No. 1242211 describes pharmaceutical combination products comprising sodium cromoglycate and isoprenaline sulphate as active ingredients and wherein the particle size of each of the active ingredients is in the range of from 1 to 10 μm.

European Patent No. 0 663 815 describes an inhalation powder which comprises a micronised active substance and a pharmaceutically acceptable excipient wherein the excipient contains a coarse fraction having an average particle size of 20 μm or more and a fine fraction with an average particle size of 10 μm or less.

International Patent Application No. WO 01/60341, which is an intervening publication, describes a powder formulation, for administration by inhalation, which comprises a mixture of an active substance which has a particle size distribution of 0.5 to 10 μm and an excipient which has a particle size distribution of from 15 to 500 μm.

International Patent Application No. WO 01/51030, which is a further intervening publication, describes a ‘bimodal’ formulation which comprises fine particles for delivery to the lung and coarse particles for delivery to the GI tract. However, such bimodal compositions do not offer any particular improvement in inhalation therapies per se.

We have now surprisingly found that the administration of a combination of active ingredients each of which has different particle sizes may be advantageous. In particular, we have found that a combination therapy comprising at least two active ingredients and wherein a first active ingredient substantially comprises a coarse fraction and a second active ingredient substantially comprises a fine fraction is especially useful in the treatment of respiratory disorders.

Thus, according to the invention we provide a bimodal pharmaceutical composition comprising effective amounts of a first active ingredient which substantially comprises a coarse fraction and a second active ingredient which substantially comprises a fine fraction characterised in that the coarse fraction possesses a greater mass median aerodynamic diameter (MMAD) than the fine fraction.

Particle size is commonly defined using mass median aerodynamic diameter (MMAD). Thus, hereinafter any reference to specific particle sizes should be construed as meaning MMAD unless otherwise defined as, for example, aerodynamic diameter. Although the sizes of the coarse and fine particles may vary, it should be understood that the coarse fraction possesses a greater MMAD than the fine fraction. That is, the majority, by mass, of the particles in the coarse fraction posses greater aerodynamic diameters than the majority of particles of the fine fraction.

Provided that the composition is bimodal as hereinbefore described, the aerodynamic particle size of the coarse fraction may be from 4 to 20 μm, preferably from 4 to 12 μm e.g. 6 μm. That is, at least 50% w/w of the particles have an aerodynamic particle diameter 6 μm. The aerodynamic particle size of the substantially fine fraction may be from 1 to 4 μm, e.g. 1 μm. That is, at least 50% w/w of the particles have an aerodynamic particle size of 1 μm.

Further, it is within the scope of this invention to include polymodal combination compositions, e.g. trimodal combinations.

The substantially coarse fraction preferentially comprises an agent which is active in the central/upper airways of a patient, e.g. the throat and/or oral cavity whilst the substantially fine fraction may comprise an agent which is active in the lung periphery.

In one embodiment of the invention the composition of the invention may be utilised in the treatment of any disorders known to be affected by corticosteroids and/or β-agonists. Thus for example the pharmaceutical composition can be useful in the treatment of non-endrocrine disorders including allergy, anaphylaxis, arteritis, collagenosis, blood disorders, cardiovascular disorders, gastro-intestinal disorders, hypercalcaemina, muscular disorders, ocular disorders, renal disorders, respiratory disease, rheumatic disorders and skin disorders.

In a preferred embodiment of the invention the pharmaceutical composition is useful, inter alia, in the treatment of respiratory disorders. In such a composition the substantially fine fraction preferentially may comprise an anti-inflammatory medicament, such as a corticosteroid, whilst the substantially coarse fraction may comprise a bronchodilator.

The substantially coarse fraction preferentially comprises a medicament which is active in the central/upper airways of a patient, such as a bronchodilator, a mucolytic agent, an antibiotic, etc.

The bronchodilators used in the composition of the invention may be selected from, but are not limited to, β₂-agonists, e.g. fenoterol, formoterol, pirbuterol, reproterol, rimiterol, salbutamol, salmeterol and terbutaline; non-selective beta-stimulants such as isoprenaline; xanthine bronchodilators, e.g. theophylline, aminophylline and choline theophyllinate; anticholinergics, e.g. ipratropium bromide; isomers and/or combinations thereof.

The corticosteroids used in the composition of the invention may be selected from, but are not limited to, beclomethasone dipropionate, fluticasone, budesonide, flunisolide, ciclesonide, triamcinolone, e.g. the acetonide, and mometasone; isomers and/or combinations thereof.

Specific combinations of medicaments which may be mentioned include combinations of steroids, such as, beclomethasone dipropionate and formoterol; beclomethasone dipropionate and salmeterol; fluticasone and formoterol; fluticasone and salmeterol; budesonide and formoterol; budesonide and salmeterol; flunisolide and formoterol; and flunisolide and salmeterol. It is also within the scope of this invention to include combinations of one or more of the aforementioned steroids with one or more of the aforementioned β₂-agonists.

The most preferred composition of the invention is one which comprises a combination of fluticasone, or a pharmaceutically acceptable ester thereof, e.g. the propionate ester, and formoterol, or a pharmaceutically acceptable salt thereof.

In the bronchodilator/corticosteroid combination composition it is preferable that the substantially coarse fraction comprises the bronchodilator and the substantially fine fraction comprises the corticosteroid.

Alternatively, the composition of the invention may deliver one or more systemically active medicaments, in which case the substantially coarse fraction may comprise, for example, a bronchodilator and the fine fraction may comprise an active agent, such as an antibiotic or a large macromolecule. Examples of such large macromolecules include, but are not limited to polypeptides, such as, insulin, growth hormone, leuprolide, interferon, parathyroid hormone and the like; and analgesic compounds, such as morphine, M6G and fentanyl.

The substantially fine fraction and/or the substantially coarse fraction may, for example, also include an absorption enhancer.

Alternatively, or in addition, the substantially coarse fraction may also include a signalling agent, for example, a flavouring agent. The term flavouring agent should be construed so as to include sweetening agents. Any conventionally known flavouring agents may be used. Such flavouring agents include, but are not limited to, peppermint oil, menthol, sugar, aspartame, cyclamates and saccharin, and salts thereof, or any combination of the aforesaid.

Therefore, in this embodiment of the invention the substantially coarse fraction may comprise a signalling agent and an active ingredient which is active in the central/upper airways of a patient, whilst the substantially fine fraction may comprise an agent which is active in the lung periphery.

Thus according to a further feature of the invention we provide a pharmaceutical composition as hereinbefore described which comprises a substantially fine fraction comprising a first active ingredient and a substantially coarse fraction comprising a signalling agent and a second active ingredient.

When the substantially coarse fraction comprises a signalling agent and a second active ingredient, the signalling agent and the second active ingredient may comprise particles of substantially similar aerodynamic particle sizes.

Alternatively, the signalling agent may comprise particles which are substantially of greater aerodynamic particle size than the second active ingredient. Thus, although not essential, such compositions may optionally be in the form of a trimodal composition.

The preferred pharmaceutical composition of the invention is most advantageous in the treatment of respiratory disorders and especially asthma and chronic obstructive pulmonary disease (COPD).

In the treatment of respiratory and/or systemic disorders the pharmaceutical composition may be delivered to the respiratory tract. Thus, delivery to the respiratory tract may comprise buccal delivery, nasal delivery or delivery by inhalation. The preferred mode of delivery to the respiratory tract is by inhalation into the lungs. Thus, the pharmaceutical composition can be administered by way of an inhaler, e.g. a metered dose inhaler or a dry powder inhaler, an insufflator or nebuliser, or any other conventionally known methods of administering inhalable medicaments.

When administered by way of inhalation the pharmaceutical composition may be in the form of a pressurised aerosol. Thus, according to a further feature of the invention we provide a pharmaceutical formulation suitable for administration by way of a pressurised aerosol comprising a pharmaceutical composition as hereinbefore described in admixture with at least a suitable propellant and optionally with a surfactant or a mixture of surfactants. The propellant is preferably a non-CFC propellant, such as a hydrofluoroalkane (HFA). Any conventionally known HFA propellant may be used, including those disclosed in, for example, EP0372777, WO91/04011, WO91/11173, WO91/11495 and WO91/14422. However, the most preferred HFA is a fluoroalkane such as a fluoromethane or a fluoroethane or a mixture of fluoroalkanes. Such fluoroalkanes include, but are not limited to, trichlorofluoromethane, dichlorodifluoromethane, 1,2-dichlorotetrafluorethane, trichlorotrifluoroethane and chloropentafluoroethane. The most preferred is HFA 134 (1,1,1,2-tetrafluoroethane) or HFA 227. The amount of propellant present may vary, but generally the pharmaceutical composition to propellant ratio will be from 1 to 300 to 1 to 5. Mixtures of propellants may also be used, for example, a mixture of HFA 134 and HFA 227. The aerosol composition of the invention may be as a solution or a suspension of the active ingredient with a propellant.

The pressurised aerosol formulation of the invention may be administered in any conventionally known inhalation apparatus.

In another embodiment the pharmaceutical composition may be administered as a dry powder formulation. Thus, according to the invention we provide a pharmaceutical formulation suitable for administration by way of a dry powder inhaler comprising a pharmaceutical composition as hereinbefore described optionally in admixture with a suitable adjuvant, diluent or carrier. When the formulation does include an adjuvant, diluent or carrier, any conventionally used ingredients in dry powder formulations may be used, such as sugars, these include, but are not limited to, dextran, mannitol and lactose, e.g. α-lactose monohydrate. Preferably, the pharmaceutical composition to carrier ratio is from 0.01:1 to 50:1.

The dry powder formulation of the invention may be administered in any conventionally known inhalation apparatus.

In a dry powder inhaler the substantially coarse fraction and the substantially fine fraction may be administered simultaneously, sequentially or separately.

However, preferred apparatus are those commercially available as CLICKHALER which is described in International Patent Application No. WO 92/00771 and/or TECHNOHALER which is described in International Patent Application No. WO 93/16748.

Alternatively, the formulation may be administered by way of a conventional nebuliser. A suitable nebuliser formulation consists of a suspension of a pharmaceutical composition of the invention in finely divided form in a sterile isotonic solvent. The suspension may be nebulised by an air jet, dropping onto an ultrasonic vibrating plate, forcing through small orifices or other known types of nebuliser, including unit-dose nebulisers, including those described by Dolovich, M., “New Propellant-free Technologies under Investigation”, J. Aerosol Medicine, 1999; 12 (suppl 1): S9-S17, such as, Respimat (from Boehringer Ingelheim), AERx™ (from Aradigm), and AeroDose (from Aerogen).

For inhalation therapy the pharmaceutical composition is preferably micronised or reduced in size by other recognised mechanisms, such as spray drying, co-milling, etc.

The dosage of pharmaceutical composition administered to a patient may vary depending, inter alia, upon the nature and severity of the disorder being treated and the method of administration. For compositions administered by inhalation therapy, the amount of the pharmaceutical composition administered may vary, depending upon, inter alia, the nature of the pharmaceutical, the disorder to be treated, the mode of administration, etc. Thus, for example, when the pharmaceutical includes an antibiotic or when the mode of administration is, by nebuliser, then the dosage is preferably in the range of from 1 μg to 500 mg. This may be 1 μg to 500 mg per metered dose or actuation or, alternatively, 1 μg to 500 mg from a plurality of metered doses or actuations. Alternatively, especially when other modes of administration are used the dosage may be in the range of from 1 μg to 300 mg, more preferably from 1 μg to 20 mg and especially from 1 μg to 5 mg.

In an especially preferred embodiment each metered dose or actuation of the inhaler will generally contain from 3 μg to 200 μg of a coarse fraction, e.g. a bronchodilator, preferably from 3 to 50 μg; and from 20 μg to 1,000 μg of a fine fraction, e.g. a corticosteroid, preferably from 20 to 500 μg. The frequency of administration of the pharmaceutical composition of the invention will vary, but most preferably, the pharmaceutical composition will be administered once or twice daily.

According to the invention we provide a method of delivering a therapeutically effective amount of a substantially fine active ingredient to the lung of a patient by the co-administration with a substantially coarse active ingredient. In the method of delivery of the invention the substantially coarse and substantially fine fractions may be administered simultaneously, sequentially or separately.

In a preferred embodiment the substantially coarse fraction is delivered to the central or upper airways of a patient and the substantially fine fraction is delivered to the lung periphery. In the most preferred embodiment the coarse and fine fractions are delivered simultaneously as a single composition as hereinbefore described. Alternatively, particularly if the coarse fraction comprises, for example, a bronchodilator, the coarse and fine fractions maybe delivered sequentially. Thus, for example, the method may comprise the administration of the coarse fraction, followed by the sequential administration of the fine fraction.

According to a further feature of the invention we provide a method of treating a respiratory disorder which comprises the simultaneous, sequential or separate administration of a therapeutically effective amount of a substantially fine fraction of an anti-inflammatory agent and a substantially coarse fraction of a bronchodilator to a patient suffering from such a disorder.

In a further method of the invention the substantially fine fraction may comprise a macromolecule as hereinbefore described, an antibiotic, a mucolytic agent, etc., optionally in combination with an absorption enhancer.

When a signalling agent is included, the signalling agent may be administered simultaneously, sequentially or separately with the active ingredients. Alternatively, the signalling agent may be delivered simultaneously with one or other of the coarse or fine fractions, whilst being delivered separately or sequentially with the other of the coarse or fine fraction. Since the signalling agent is itself preferentially comprised of substantially coarse particles, then in a preferred embodiment of the invention the signalling agent may be administered simultaneously with the coarse fraction.

We further provide a method of treating COPD Which comprises the simultaneous, sequential or separate administration of a therapeutically effective amount of a corticosteroid and a bronchodilator to a patient suffering from such a disorder.

In the methods of the invention, the anti-inflammatory agent and the bronchodilator may be administered as separate compositions, which may be administered simultaneously, sequentially or separately or as a single combination product. Each metered dose or actuation of the inhaler will generally contain from 3 μg to 50 μg of the bronchodilator and from 20 μg to 500 μg of the anti-inflammatory agent. The frequency of administration of the pharmaceutical composition of the invention will vary, but most preferably, the pharmaceutical composition will be administered once or twice daily in, for example, the treatment of asthma.

In a preferred embodiment, the method of treatment of the invention comprises the administration of a therapeutically effective amount of a corticosteroid and a bronchodilator as a pharmaceutical composition as hereinbefore described.

We also provide the use of an anti-inflammatory agent in the manufacture of a pharmaceutical composition as hereinbefore described.

Alternatively we provide the use of a bronchodilator in the manufacture of a pharmaceutical composition as hereinbefore described.

We especially provide the use of a mixture of an anti-inflammatory agent and a bronchodilator in the manufacture of a pharmaceutical composition as hereinbefore described.

In a bronchodilator/corticosteroid combination therapy, the ratio of bronchodilator to corticosteroid in the composition according to the invention may vary, but is preferably within the range from 1:0.4 to 1:167.

We further provide a process for the manufacture of a pharmaceutical composition as hereinbefore described which comprises mixing a substantially coarse fraction of an active agent with a substantially fine fraction of an active agent, and optionally at the same time or sequentially mixing a pharmaceutically acceptable adjuvant, diluent or carrier.

A variety of medicaments may be administered in simultaneously, sequentially or separately with the composition of the invention. Such medicaments are generally antibiotics, bronchodilators or other anti-asthma drugs. Such medicaments include, but are not limited to β₂-agonists, e.g. fenoterol, formoterol, pirbuterol, reproterol, rimiterol, salbutamol, salmeterol and terbutaline; non-selective beta-stimulants such as isoprenaline; xanthine bronchodilators, e.g. theophylline, aminophylline and choline theophyllinate; anticholinergics, e.g. ipratropium bromide; mast cell stabilisers, e.g. sodium cromoglycate and ketotifen; bronchial anti-inflammatory agents, e.g. nedocromil sodium; and steroids, e.g. beclomethasone dipropionate, fluticasone, budesonide, ciclesonide, triamcinolone, e.g. the acetonide, and flunisolide; and combinations thereof.

The invention will now be illustrated but shall not be limited to the following example.

### EXAMPLE 1

### Formulation

A bimodal dry powder inhalation formulation was prepared comprising:

as coarse fraction—6 μg formoterol fumarate with particle size 5-6 μm

as fine fraction—100 μg fluticasone propionate with particle size 2-3 μm

What is claimed:

1. A bimodal pharmaceutical composition comprising:
effective amounts of a first active ingredient which substantially comprises a coarse fraction; and
a second active ingredient which substantially comprises a fine fraction;
characterised in that the coarse fraction possesses a greater mass median aerodynamic diameter (MMAD) than the fine fraction.

2. A bimodal pharmaceutical composition according to claim 1 characterised in that the aerodynamic particle size of the substantially coarse fraction is from 4 to 20 μm.

3. A bimodal pharmaceutical composition according to claim 2 characterised in that at least 50% w/w of the coarse particles have an aerodynamic particle size of from 4 to 20 μm.

4. A bimodal pharmaceutical composition according to claim 3 characterised in that the aerodynamic particle size of a substantial amount of the coarse fraction is 6 μm.

5. A bimodal pharmaceutical composition according to claim 1 characterised in that the aerodynamic particle size of the substantially fine fraction is from 1 to 4 μm.

6. A bimodal pharmaceutical composition according to claim 5 characterised in that at least 50% w/w of the fine particles have an aerodynamic particle size of from 1 to 4 μm.

7. A bimodal pharmaceutical composition according to claim 6 characterised in that the aerodynamic particle size of a substantial amount of the fine fraction is 1 μm.

8. A bimodal pharmaceutical composition according to claim 1 characterised in that the pharmaceutical composition is suitable for the treatment of one or more disorders selected from allergy, anaphylaxis, arteritis, collagenosis, blood disorders, cardiovascular disorders, gastro-intestinal disorders, hypercalcaemia, muscular disorders, ocular disorders, renal disorders, respiratory disease, rheumatic disorders and skin disorders.

9. A bimodal composition according to claim 1 characterised in that the composition includes a signaling agent.

10. A bimodal composition according to claim 9 characterised in that the signaling agent is comprised in the coarse fraction.

11. A bimodal composition according to claim 10 characterised in that the coarse signaling agent creates a trimodal composition.

12. A bimodal pharmaceutical composition according to claim 8 characterised in that the pharmaceutical composition is suitable for the treatment of respiratory disorders.

13. A bimodal pharmaceutical composition according to claim 12 characterised in that the substantially coarse fraction comprises an agent which is active in the central/upper airways of a patient.

14. A bimodal pharmaceutical composition according to claim 12 characterised in that the substantially fine fraction comprises an agent which is active in the lung periphery.

15. A bimodal pharmaceutical composition according to claim 12 characterised in that the substantially fine fraction comprises an anti-inflammatory agent.

16. A bimodal pharmaceutical composition according to claim 12 characterised in that the substantially coarse fraction comprises a bronchodilator.

17. A bimodal pharmaceutical composition according to claim 15 characterised in that the substantially fine fraction comprises an anti-inflammatory agent and the substantially coarse fraction comprises a bronchodilator.

18. A bimodal pharmaceutical composition according to claim 15 characterised in that the anti-inflammatory agent is a corticosteroid.

19. A bimodal pharmaceutical composition according to claim 18 characterised in that the corticosteroid is selected from one or more of beclomethasone, fluticasone, budesonide, flunisolide, ciclesonide, triamcinolone, and mometasone, and pharmaceutically acceptable esters thereof.

20. A bimodal pharmaceutical composition according to claim 17 characterised in that the composition comprises a combination of fluticasone, or a pharmaceutically acceptable ester thereof, and formoterol, or a pharmaceutically acceptable salt thereof.

21. A pharmaceutical composition according to claim 1 characterised in that at least one of the active ingredients is systemically active in a patient.

22. A bimodal pharmaceutical composition according to claim 21 characterised in that the substantially fine active ingredient is systemically active in a patient.

23. A bimodal pharmaceutical composition according to claim 22 characterised in that the substantially fine fraction is selected from one or more of, an antibiotic and a macromolecular medicament.

24. A bimodal pharmaceutical composition according to claim 23 characterised in that the macromolecular medicament is selected from one or more polypeptides.

25. A bimodal pharmaceutical composition according to claim 24 characterised in that the macromolecule is selected from insulin, growth hormone, leuprolide, interferon and parathyroid hormone.

26. A bimodal pharmaceutical composition according to claim 23 characterised in that the macromolecular medicament is an analgesic compound.

27. A bimodal pharmaceutical composition according to claim 26 characterised in that the analgesic compound is selected from morphine, M6G and fentanyl.

28. A bimodal pharmaceutical composition according to claim 22 characterised in that the composition includes an absorption enhancer.

29. A bimodal pharmaceutical formulation according to claim 12 suitable for administration by way of a pressurised aerosol comprising such a pharmaceutical composition in admixture with at least a suitable propellant.

30. A bimodal pharmaceutical composition according to claim 12 suitable for administration by a dry powder inhaler comprising such a pharmaceutical composition.

31. A bimodal pharmaceutical composition according to claim 30 characterised in that the composition includes a pharmaceutically acceptable adjuvant, diluent or carrier.

32. A bimodal pharmaceutical formulation according to claim 31 characterised in that the pharmaceutical composition to carrier ratio is from 0.01:1 to 50:1.

33. A dry powder inhaler containing a pharmaceutical composition comprising:
effective amounts of a first active ingredient which substantially comprises a coarse fraction; and
a second active ingredient which substantially comprises a fine fraction; which fractions may be administered simultaneously, sequentially or separately.

34. A dry powder inhaler according to claim 33 characterised in that the inhaler is a dry powder inhaler for delivering a substance in a finely divided form, comprising:
a body defining a storage chamber for the substance to be delivered and further defining an inhalation passage through which air is drawn via a mouthpiece;
a metering member operable to transfer a volumetric dose of the substance from the storage chamber to the inhalation passage, the metering member having a metering surface which is indented to provide at least one dispensing cup and being moveable between a first position in which a dispensing cup is presented to the storage chamber to receive a dose of the substance and a second position in which a dose of the substance is presented to the inhalation passage in a position which is upwardly open in use; and
means for ensuring that each dispensing cup is substantially free from the substance before being presented to the storage chamber.

35. A dry powder inhaler according to claim 33 characterised in that the inhaler is a dry powder inhaler for delivering a substance in a finely divided form, the inhaler comprising:
a storage chamber for storing a quantity of the substance to be delivered;
air intake means by which air may be drawn into the inhaler from the atmosphere;
an inhalation passage communicating with the air intake means, through which passage air may be drawn using the air intake means;
a storage chamber outlet conduit communicating between the storage chamber and the inhalation passage;
a metering device for use in transferring a desired volumetric dose of the substance from the storage chamber to the inhalation passage via the outlet conduit, the metering device being movable through the outlet conduit from a first position, in which it is presented to the storage chamber to receive the substance, to a second position in which the desired volumetric dose of the substance is presented with the metering device to the inhalation passage; and
indexing means operable to move the metering device from its first to its second position.

36. A bimodal pharmaceutical composition according to claim 12 characterised in that a single dosage administrable to a patient is in the range of from 1 μg to 300 mg.

37. A bimodal pharmaceutical composition according to claim 12 characterised in that a single dosage administrable to a patient comprises from 3 to 200 μg of the coarse fraction and from 20 to 1,000 μg of the fine fraction.

38. A bimodal pharmaceutical composition suitable for administration by way of a nebuliser comprising a suspension of a pharmaceutical composition according to claim 1.

39. A bimodal pharmaceutical composition according to claim 38 characterised in that the dosage administered is in the range of from 1 μg to 500 mg.

40. A method of delivering a therapeutically effective amount of a substantially fine active ingredient to the lung of a patient by the co-administration with a substantially coarse active ingredient.

41. A method according to claim 40 characterised in that it includes the simultaneous, sequential or separate administration of a signaling agent.

42. A method according to claim 40 characterised in that the active ingredients are delivered by way of inhalation.

43. A method according to claim 40 characterised in that the substantially coarse fraction is delivered to the central or upper airways of a patient and the substantially fine fraction is delivered to the lung periphery.

44. A method of treating a respiratory disorder which comprises the simultaneous, sequential or separate administering of a therapeutically effective amount of a substantially coarse fraction of an anti-inflammatory agent and a substantially fine fraction of a bronchodilator to a patient suffering from such a disorder.

45. A method according to claim 44 characterised in that the coarse and fine fractions are administered as a single composition.

46. A method according to claim 44 characterised in that the substantially coarse fraction includes a signaling agent.

47. A method of treating COPD which comprises the simultaneous, sequential or separate administering of a therapeutically effective amount of a substantially fine fraction of a corticosteroid and a substantially coarse fraction of a bronchodilator to a patient suffering from such a disorder.

48. A method of treatment according to claim 40 characterised in that the method comprises the administration of a therapeutically effective amount of a corticosteroid and a bronchodilator as a pharmaceutical composition.

49. A bimodal pharmaceutical composition according to claim 17 characterised in that the ratio of bronchodilator to anti-inflammatory agent is within the range from 1:0.4 to 1:167.

50. A process for the manufacture of a bimodal pharmaceutical composition according to claim 1 which comprises mixing a substantially coarse fraction of an active agent with a substantially fine fraction of an active agent, and optionally at the same time or sequentially mixing a pharmaceutically acceptable adjuvant, diluent or carrier.

51. A bimodal pharmaceutical composition according to claim 17 characterised in that the composition comprises a combination of beclomethasone dipropionate and formoterol; beclomethasone dipropionate and salmeterol; fluticasone and formoterol; fluticasone and salmeterol; budesonide and formoterol; budesonide and salmeterol; flunisolide and formoterol; or flunisolide and salmeterol.

52. A bimodal pharmaceutical composition according to claim 17 characterised in that the composition comprises a combination of fluticasone or a pharmaceutically acceptable ester thereof, and salmeterol.
